(19) United States
(12) Patent Application Publication
(10) Pub. No.: US 2004/0225197 A1
(43) Pub. Date: Nov. 11, 2004

(54) Surgical retractor

(76) Inventors: Daniel Roux, Rouffiac Tolosan (FR); Anthony Paolitto, St. Leonard (CA); Valerio Valentini, Montreal (CA)

Correspondence Address: ANTHONY PAOLITTO, CORONEO, INC., Suite 514, 9250 Avenue du Parc, Montreal, H2N 1Z2, CA

(21) Appl. No.: 10/838,333

(22) Filed: May 5, 2004

Related U.S. Application Data
(60) Provisional application No. 60/467,579, filed on May 5, 2003.

(51) Int. Cl. A61B 1/32

(52) U.S. Cl. 600/231

(57) ABSTRACT

A surgical retractor for retracting a pair of opposed tissue edges to define a surgical opening extending in a surgical opening plane. The retractor consists of a pair of tissue blades configured to contact the tissue edges, an actuator releasably attached to the tissue blades for selective adjustment of the spacing between the blades, and a bracing member attachable to the tissue blades for maintaining the the blades in a desired retracted configuration when the actuator is separated from the blades.

This application claims the benefits of U.S. Provisional Patent Application Ser. No. 60/467,579, filed on May 5, 2003.

### FIELD OF THE INVENTION

The present invention relates to the general field of surgical accessories and is particularly concerned with a surgical retractor.

### BACKGROUND OF THE INVENTION

As is well known, during surgery, it is frequently necessary to retract tissue, bone or other body material in order to facilitate access to and visualization of the surgical site. In thoracic surgery such as cardiac surgery, a median sternotomy is frequently performed to allow access to organs such as the heart within the thoracic cavity.

As is well known, the sternum, also commonly referred to as the breast bone, is a relatively short bone in the middle of the chest interconnecting ribs 1 through 7 and to which all the ribs are attached either directly or indirectly. During standard median sternotomy, it is now standard procedure to access the thoracic cavity by initially performing a medial incision down the centre of the chest with a cauterizing scalpel, beginning at the base of the throat and cutting down past the medial portion of the sternum or breast bone. A small hand held electric saw is then used to sever the sternum.

After the incision and cutting away has been completed, the surgical team can begin to open up the sternum to get to the thoracic cavity of the patient. The gap or slit opening created by the cut in the severed sternum allows the surgeon to insert a so-called surgical retractor, oftentimes also referred to as a sternum spreader, in between the edges of the cut sternum. The surgical retractor is used to mechanically and physically spread apart and pry apart the rib cage to provide access to the thoracic cavity. Typically, in the case of thoracic surgery performed on an adult patient, the cavity is opened up about 6 to 8 inches across.

Typical prior art surgical retractors include a pair of elongated metal members, also termed arms, with blades disposed thereon to capture the sternum. Typical prior art retractors also include a bar or rack having rack teeth extending therefrom to which the arms are mechanically coupled. One of the arms is typically fixed in position on the rack while the other arm is provided with a pinion or cog gear interacting with the rack teeth so as to allow selective movement along the rack. Movement of the movable arm along the rack, in turn, changes the separation distance between the blades. For pediatric and small patients, a small sized retractor of the same general configuration as described above may be used.

During a conventional median sternotomy, once the arms of the retractor are put into contact with the edges of the cut sternum, the retractor is cranked open using a rotatable handle until the two spreader arms abutting against the generally opposed sternum edges force the sternum and rib cage to separate and expand open, thereby giving access to the thoracic cavity, and the internal tissues and body organs therewithin. Upon completion of the surgery, the severed sternal sections of the sternum are re-joined and securely closed.

For proper healing to occur, the split sternum portions must be appositioned and held together while the sternum heals. Traditional methods for closing a sternum involve securing steel wires around or through the sternum halves and approximating the sternum by twisting the wires together.

Some thoracic surgeries, such as certain types of pediatric heart surgeries, are particularly susceptible of producing oedema of the myocardial tissue and/or pericardial and/or adjacent tissue. In some situations, the oedema is such that approximation of the sternum halves must be delayed until the oedema is resolved, typically within a post-operative period of four to five days.

In order to maintain the sternum in its retracted configuration until the oedema resolves, the conventional retractors are typically removed from the incision site and replaced by less traumatic and bulky makeshift objects such as chest cannulas, tubes or the like positioned in abutting relationship with opposite edges of the sternum typically adjacent the longitudinal ends of the sternal incision. The opening in the skin formed by the incision is temporarily closed by suturing a patch, typically made out of synthetic material, across the opening.

Although useful and widely accepted for most thoracic surgeries, conventional surgical retractors nevertheless suffer from numerous drawbacks or shortcomings that are manifested and become apparent both during the operative and post-operative periods.

Typically, the sternum is spread apart using either curved or straight blades. The curved blades, also commonly referred to as “Cooley” style blades, are preferred by some surgeons since they provide the latter with better stabilization of the retractor in the chest incision since the sternum is captured within the curvature of the blade. One of the problems associated with these types of blades is that they are more difficult to insert into the severed or incised sternum because they have a larger side profile.

The other type of commonly used blades are the straight blades. These are generally referred to as “Finochietto” style blades. The straight blades are preferred by some surgeons because they have a low side profile and are easy to insert into the chest incision. One of the drawbacks associated with straight blades is that they offer the surgeon less stabilization of the retractor in the chest incision than the curved blades.

A drawback associated with conventional retractors having both straight and curved blades is that since a relatively large force is needed (typically up to 100 pounds acting on each of the blades) in order to retract the sternum halves away from each other, a relatively large pressure is typically created between the sternum blades and the body tissue during retraction thereof. This relatively large pressure is sometimes traumatic to the body tissue.

Furthermore, some prior art retractors have also been linked to transitory neuropathy potentially related to the fact that conventional surgical retractors produce substantial pressure on the lower branch of the brachial plexus. This type of injury to the brachial plexus can be related to the fact that the retractor arms typically remain parallel with respect to each other throughout their range of motion.

One method of overcoming the problem of applying excessive pressure to the upper ribs and the adjacent portions of the brachial plexus has been to attempt positioning of the retractor as low as possible so that there is minimal pressure on the upper, shorter ribs. However, this approach is often not desirable since the surgeon is not able to position the retractor in the most advantageous position for retraction of the chest.

Another problem associated with prior art surgical retractors is that the rack and pinion mechanism used for moving the arms relative to one another is relatively bulky and cumbersome. The moving member containing the cog gear and the handle attached thereto in particular not only often causes visual obstruction of the surgical site but also often blocks at least partially physical access to the surgical site from certain angles and has been associated with tangling therewith of surgical wires and instruments.

The problems associated with potential trauma to body tissue and both physical and visual obstruction of the surgical field is compounded and becomes both more prevalent and critical in the case of pediatric surgeries. Indeed, although some components of conventional surgical retractors are downsized for pediatric usage, other components are difficult to scale down and, hence, pediatric surgical retractors are often considered more problematic.

Furthermore, conventional surgical retractors and, in particular, pediatric retractors are unsuitable in situations such as hereinabove disclosed, requiring the sternal half sections to remain temporarily spread apart during part of the post-operative period, sometimes for days, until the oedema within the thoracic cavity resolves. For example, if the retracted sternum needs to be closed with a patch, portions of the existing conventional retractors extend beyond and/or above the sternal opening, and as such render very difficult, impracticable, or impossible the patching over of the sternal opening.

Accordingly, there exists a need for an improved surgical retractor.

### SUMMARY OF THE INVENTION

It is a general object of the present invention to provide such an improved surgical retractor.

Advantages of the present invention include that the proposed surgical retractor allows for safe and relatively atraumatic retraction of body tissue. More specifically, in at least one embodiment of the invention, the proposed surgical retractor is designed so as to reduce the pressure exerted on body tissues in contact therewith for a given spreading force imparted thereon.

Also, in at least one embodiment of the invention, the surgical retractor is designed so as to allow for an angled movement as opposed to a parallel movement between the spreader arms during the spreading operation so that the arms form a generally triangularly shaped opening in the chest as the sternum is spread apart in order to reduce the risks of brachial plexus injury and the risk of broken ribs, particularly the shorter ribs.

Also, the proposed surgical retractor is designed so as to reduce both visual and physical obstruction to the surgical site throughout the use thereof. More specifically, in at least one embodiment of the invention, the driving mechanism used for moving the surgical arms and blades relative to each other may be readily removed from the blades once they are sufficiently spread apart.

Bracing means are provided for maintaining the blades in a spread apart relationship relative to each other once the driving mechanism has been removed. The removal of the driving mechanism from and attachment of the bracing means to the blades is typically allowed through a set of quick and ergonomic steps.

In at least one other embodiment of the invention, reduction of both visual and physical obstruction to the surgical site throughout the use thereof is achieved by providing a combined driving/bracing means designed so as to be located below the geometrical plane formed by the blades once the latter are in a retracted configuration.

Also, in at least one embodiment of the invention, the surgical retractor is designed so as to be suitable for maintaining the surgical incision opened during part of the post-operative period with reduced risks of associated injury while allowing mounting thereto or adjacent thereto of a temporary closing membrane such as an artificial skin patch again through a set of quick and ergonomic steps.

Furthermore, the proposed surgical retractor is designed so as to be structurally simple and manufacturable through conventional forms of manufacturing so as to provide a surgical retractor that will be economically feasible, long-lasting and relatively trouble-free in operation. In at least one embodiment, the proposed surgical retractor is fabricated from lightweight surgical grade material, such as titanium, which provides a weight advantage when such retractor is used in pediatric surgery, and a low weight retractor is desirable to apply low weight on the infant's retracted sternum.

### BRIEF DESCRIPTION OF THE DRAWINGS

Various embodiments of the present invention will now be disclosed, by way of example, in reference to the following drawings in which:

FIG. 1, in a schematic perspective view, illustrates a surgical retractor in accordance with an embodiment of the present invention, the surgical retractor being shown retracting a pair of tissue edges and about to receive one of a set of differently sized spacing braces;

FIG. 2, in a schematic perspective view, illustrates the surgical retractor shown in FIG. 1, with its driving mechanism removed therefrom and with a spacing brace part of the invention being used for maintaining spreader arms in a spread apart relationship relative to each other for spreading apart the tissue edges;

FIG. 3a, in a partial longitudinal cross-sectional view taken along the cross-sectional arrows of FIG. 1, illustrates the relationship between the driving mechanism and one of the spreader arms when the latter are coupled together;

FIG. 3b, in a partial longitudinal cross-sectional view and similar to that of FIG. 3a, illustrates part of the driving mechanism being separate from a corresponding spreader arm, both part of a surgical retractor in accordance with an embodiment of the present invention;

FIG. 4a, in a top view, illustrates an alternative type of connection between a part of a driving mechanism and a spreader arm, both part of a surgical retractor in accordance with an alternative embodiment of the invention with the spreader arm mechanically coupled to the driving mechanism;

FIG. 4b, in a longitudinal cross-sectional view taken along arrows 4b-4b, illustrates the relationship between part of the driving mechanism and the spreader arm shown in FIG. 4a;

FIG. 4c, in a transversal cross-sectional view taken along arrows 4c-4c of FIG. 4b, illustrates the relationship between coupling sections of the blade and part of the drive mechanism shown in FIG. 4a;

FIG. 4d, in a transversal cross-sectional view similar to that of FIG. 4c, illustrates an alternative embodiment of the relationship between alternative configurations of the coupling components between the drive mechanism and retractor blade shown in FIGS. 4a through 4c;

FIG. 5a, in a partial perspective view with sections taken out, illustrates coupling sections of a spacing brace and a spreader arm, both part of a surgical retractor in accordance with an embodiment of the present invention as the components are about to be assembled together;

FIG. 5b, in a partial perspective view with sections taken out similar to that of FIG. 5a, illustrates an alternative embodiment of coupling components for coupling the spacing brace to the retractor arm;

FIG. 5c, in a partial perspective view with sections taken out similar to that of FIG. 5a, illustrates another alternative embodiment of coupling components for coupling the spacing brace to the retractor arm;

FIG. 5d, in a partial perspective view with sections taken out similar to that of FIG. 5a, illustrates yet another alternative embodiment of coupling components for coupling the spacing brace to the retractor arm;

FIG. 5e, in a partial perspective view with sections taken out similar to that of FIG. 5a, illustrates still another alternative embodiment of coupling components for coupling the spacing brace to the retractor arm;

FIG. 5f, in a partial transversal cross-sectional view taken along the cross-sectional arrows of FIG. 5e illustrates part of the spacing brace and the retractor arm assembled together in an unlocked configuration on the left-hand side and on the right-hand side in a locked configuration;

FIG. 6a, in a partial perspective view with sections taken out, illustrates part of a retractor arm in accordance with an embodiment of the present invention, the retractor arm being shown with a surgical implement mounted thereon using mounting groove formed thereon;

FIG. 6b, in a partial perspective view with sections taken out, illustrates part of a retractor arm in accordance with an embodiment of the present invention, the retractor arm being shown with a mounting protrusion extending therefrom;

FIG. 6c, in a partial perspective view with sections taken out, illustrates part of a retractor arm in accordance with an alternative embodiment of the present invention, the retractor arm being shown with a mounting protrusion having an alternate configuration extending therefrom;

FIG. 6d, in a partial perspective view with sections taken out, illustrates part of a retractor arm in accordance with yet another alternative embodiment of the present invention, the retractor arm being shown with a mounting protrusion having yet another alternate configuration extending therefrom;

FIG. 7a, in a perspective view, illustrates a longitudinally curved retractor arm part of a surgical retractor in accordance with an embodiment of the present invention;

FIG. 7b, in a transversal cross-sectional view taken arrows 7b-7b of FIG. 7a, illustrates a cross-sectional configuration of the retractor blade shown in FIG. 7a;

FIG. 7c, in a transversal cross-sectional view similar to that of FIG. 7b, illustrates an alternative cross-sectional configuration for the retractor blade;

FIG. 7d, in a transversal cross-sectional view similar to FIGS. 7b and 7c, illustrates yet another alternative cross-sectional configuration of the retractor blade shown in FIG. 7a;

FIG. 8, in a perspective view, illustrates a retractor blade in accordance with an alternative embodiment of the invention;

FIG. 9, in a perspective view, illustrates a retractor blade in accordance with an embodiment of the present invention, the blade being shown with one of its tissue contacting components about to be assembled thereto;

FIG. 10, in a perspective view, illustrates a surgical retractor in accordance with yet another alternative embodiment of the invention;

FIG. 11, in a partial perspective view with sections taken out, illustrates yet another alternative embodiment of the invention, the embodiment being shown with its driving actuating mechanism about to be assembled the retractor blade;

FIG. 12a, in a partial perspective view, illustrates yet another alternative embodiment of the invention, the surgical retractor shown in FIG. 12a being shown in a substantially closed configuration;

FIG. 12b, in a partial perspective view, illustrates the surgical retractor shown in FIG. 12a in a fully retracting configuration;

FIG. 13a, in a partial perspective view with sections taken out, illustrates part of the surgical retractor in accordance with an embodiment of the present invention with a covering patch being mounted thereto;

FIG. 13b, in a partial perspective view with sections taken out, illustrates an alternative embodiment of an attachment means used for attaching the covering patch shown in FIG. 13a to a retractor blade in accordance with an embodiment of the present invention;

FIG. 13c, in a partial perspective view with sections taken out, illustrates yet another alternative embodiment of an attachment means for attaching the covering patch to the retractor arm;

FIG. 13d, in a partial perspective view with sections taken out, illustrates yet another alternative embodiment of an attachment means for attaching the covering patch to the retractor arm;

FIG. 13e, in a partial perspective view with sections taken out, illustrates yet another alternative embodiment of an attachment means for attaching the covering patch to the retractor arm;

### DETAILED DESCRIPTION

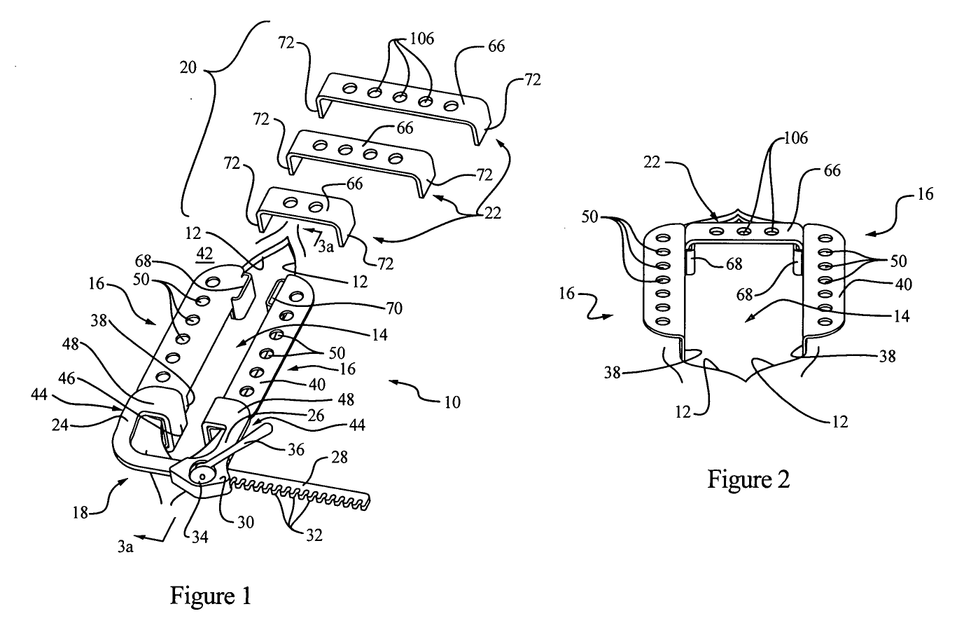

Referring to FIG. 1, there is shown a surgical retractor 10 in accordance with an embodiment of the present invention. The retractor 10 is shown being used for retracting a pair of substantially opposed body tissue edges 12 delimiting an incision opening 14. It should be understood that although the retractor 10 is disclosed herein as being used in a context of a sternotomy-type surgical intervention, the retractor 10 could be used in any other contexts including other types of thoracic surgeries and surgeries performed on other parts of a human or animal body without departing from the scope of the present invention.

According to the principle of the present invention, the retractor 10 generally involves a pair of retractor blades 16 coupled to a driving means 18 for driving the blade 16 so as to vary the distance therebetween. Typically, the driving means is a drive mechanism that functions in some manner to urge the opposing blade 16 apart thus forcing the opposite tissue edges 12 open in order to allow surgical access through the incision opening 14.

Typically, the driving means 18 is constructed to spread the opposing blades 16 apart in a generally parallel fashion. However, in other embodiments hereinafter disclosed, the parting motion may also have a significant curvilinear or angular component as well.

In accordance with one aspect of the present invention, at least a portion of at least one of the blades 16 is separable from the driving means 18, i.e. some of the features and functions associated with at least a portion of at least one of the retractor blades 16 are allocated to a structural component which is separate, separable or otherwise detachable from the driving means 18.

In accordance with another aspect of the present invention, the retractor 10 further includes a bracing means 20 for maintaining the retractor blades 16 in a retracted configuration such as shown in FIG. 2, once the driving means 18 has been detached or separated from the blades 16. In at least one embodiment of the invention, the bracing means includes at least one spacing brace 22 permanently or releasably attachable to the blades 16. In accordance with at least one embodiment of the invention (not shown) the spacing brace 16 is permanently attached to one of the blades 16 while being releasably attachable to the other or opposed blade 16.

In the embodiments shown throughout the figures, each of the blades 16 has a blade first segment 38 for substantially contacting a corresponding tissue edge 12 and a blade second segment 40 extending therefrom for substantially contacting an exterior surface 42 of the tissue in which the incision 14 is made.

The driving means 18 may take any suitable form. In at least one embodiment of the invention, the driving means includes a rack and pinion type of mechanism.

Typically, the driving means or mechanism 18 includes a first and a second arm 24, 26 mounted to a bar or rack 28 for relative movement therebetween along the rack 28. Typically, the first arm 24 is fixedly attached to the rack 28 while the second arm 26 is rigidly attached to a moving member 30 movable along the rack 28.

Typically, the rack 28 is provided with at least one row of gear teeth 32. A pinion or cog gear 34 is rotatably mounted to the moving member 30 for mechanically engaging the gear teeth 32. A rotatable handle 36 is hingedly coupled to the cog gear 34 for rotating the latter so as to cause relative movement between the first and second arms 24, 26.

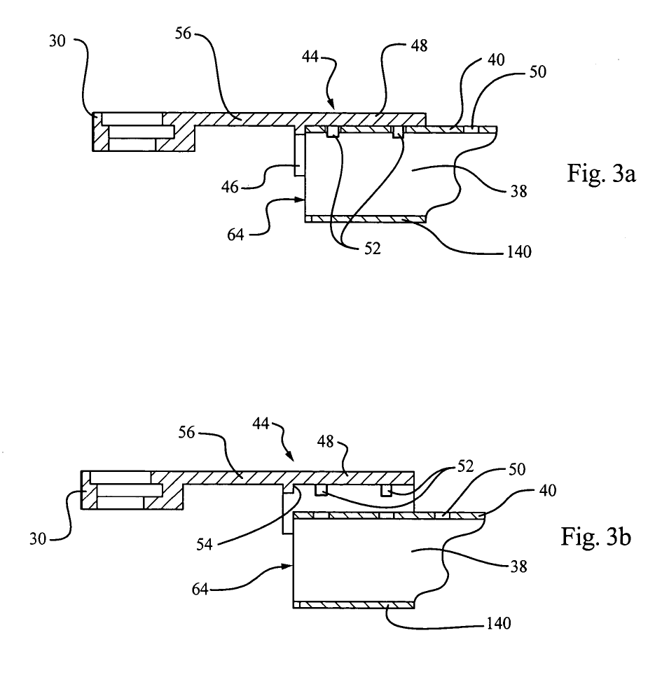

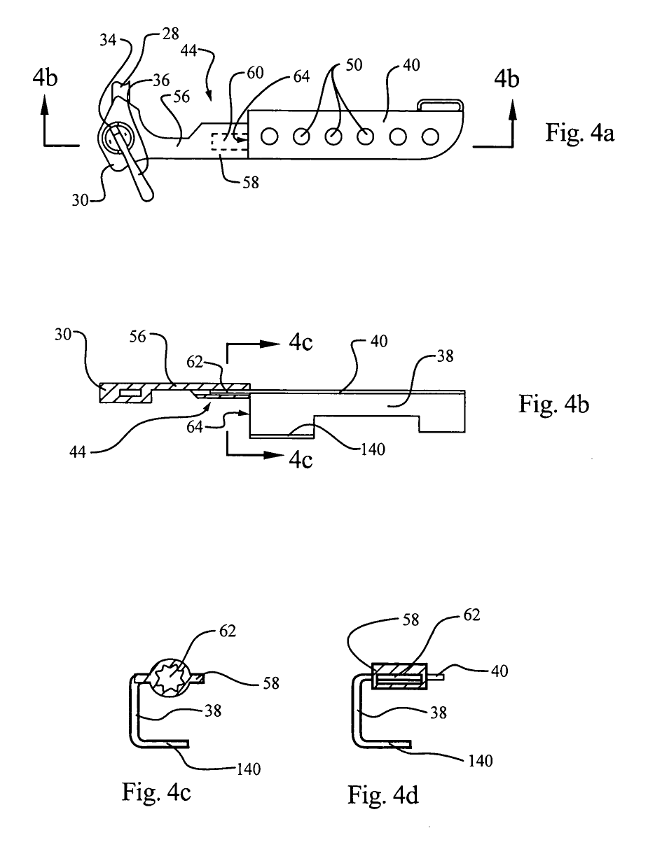

One of the features of the present invention resides in that at least one of the first or second arms 24, 26 and preferably both the first and second arms 24, 26 are releasably attachable respectively to a corresponding one of the blades 16 using a suitable arm-to-blade releasable attachment means. The arm-to-blade attachment means may include threaded connections or other mating features on the blades 16 and arms 24, 26 themselves, ordinary or specialized mechanical fasteners, cam or latching mechanisms adapted to releasably secure the blades 16 to the arms 24, 26. It should be understood that the arm-to-blade releasable attachment means may take any suitable form including a mechanical, magnetic, electromagnetic or otherwise generated operational interface without departing from the scope of the present invention. FIGS. 3a through 4d illustrate various mechanical embodiments of the arm-to-blade releasable attachment means.

In the embodiments shown in FIGS. 3a through 4d, the arm-to-blade releasable attachment means includes an arm coupling section 44 for coupling to a corresponding blade 16. In the embodiments shown in FIGS. 3a and 3b, the coupling section 44 includes a coupling section first segment 46 for contacting the blade first segment 38 and a coupling section second segment 48 for interfacing with the blade second segment 40.

Typically, the coupling section first segment 46 has a generally flat configuration for abuttingly contacting the exterior surface (tissue non-contacting surface) of the blade first segment 38 while minimally protruding into the incision opening 14. Typically, the blade second segment 40 is provided with at least one and preferably a plurality of attachment apertures 50 extending therethrough.

At least one and preferably a pair of attachment tongues or pins 52 typically extends outwardly from the coupling section second segment 48. The coupling tongues 52 are configured and sized for allowing slidable insertion thereof into corresponding attachment or coupling apertures 50. Typically, an abutment shoulder 54 is defined between the coupling section second segment 48 and a spacing segment 56 of the first and second arms 24, 26 for facilitating and guiding the coupling of the first and second arms 24, 26 to a corresponding blade 16. Alternatively, abutment shoulder 54 may be removed (not shown) and coupling section second segment 48 extended along arm 24, 26 thereby allowing blades 16 to be coupled closer to the rack 28 in a manner that pins 52 are slidingly insertable in any two successive coupling apertures 50.

In the embodiment shown in FIG. 4a, a coupling section 44 includes a coupling sleeve 58 extending outwardly from the spacing segment 56. The coupling sleeve 58 is provided with a sleeve channel 60 extending therein. The blade second segment 40 is provided with a blade coupling tongue or pin 62 extending substantially outwardly from a position located adjacent one of its longitudinal ends 64. The blade coupling tongue 62 is configured and sized for being substantially fittingly and slidably insertable into a coupling channel 60 of the coupling sleeve 58.

In the embodiment shown in FIG. 4c, the blade coupling tongue 62 and the coupling channel 60 both have a generally disc-shaped configuration. More specifically, the coupling tongue 62 and coupling channel 60 may be complementarily splined. As such, blades 16 may be selectively coupled in one of a plurality of angular orientations or relationships relative to arm 24, 26. In the embodiment shown in FIG. 4d, the blade coupling tongue 62 and the sleeve channel 60 both have a generally rectangular configuration. It should be understood that both the blade coupling tongue 62 and the sleeve channel 60 could have other configurations without departing from the scope of the present invention.

In accordance with yet another feature of the present invention, there is also provided spacing braces 22 for maintaining the blades 16 in a retracted configuration such as shown in FIG. 2 when the driving means 18 is removed or separated from the blade 16. Typically, each spacing brace 22 includes a brace spacing segment 66 for extending between the blades 16. Each spacing brace 22 typically also includes at least one and typically two brace-to-blade releasable attachment means for releasably attaching the spacing brace 22 to at least one and typically both blades 16.

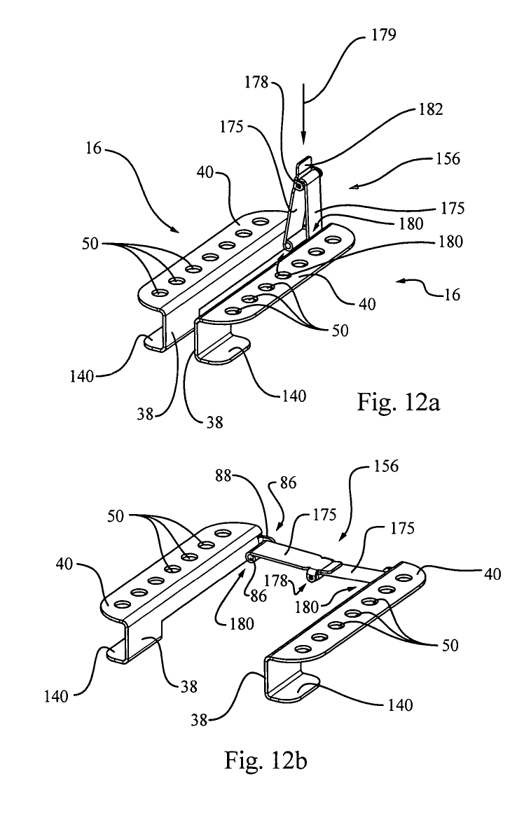

In the embodiments shown throughout most of the figures, the spacing segment 66 has a substantially fixed length. However, in the embodiments shown in FIGS. 11 and 12, the spacing segment 66 is provided with length adjustment means for allowing customization of its length. It should be understood that other types of length adjustment means such as telescopically coupled sections of spacing segment 66 or the like could be used without departing from the scope of the present invention.

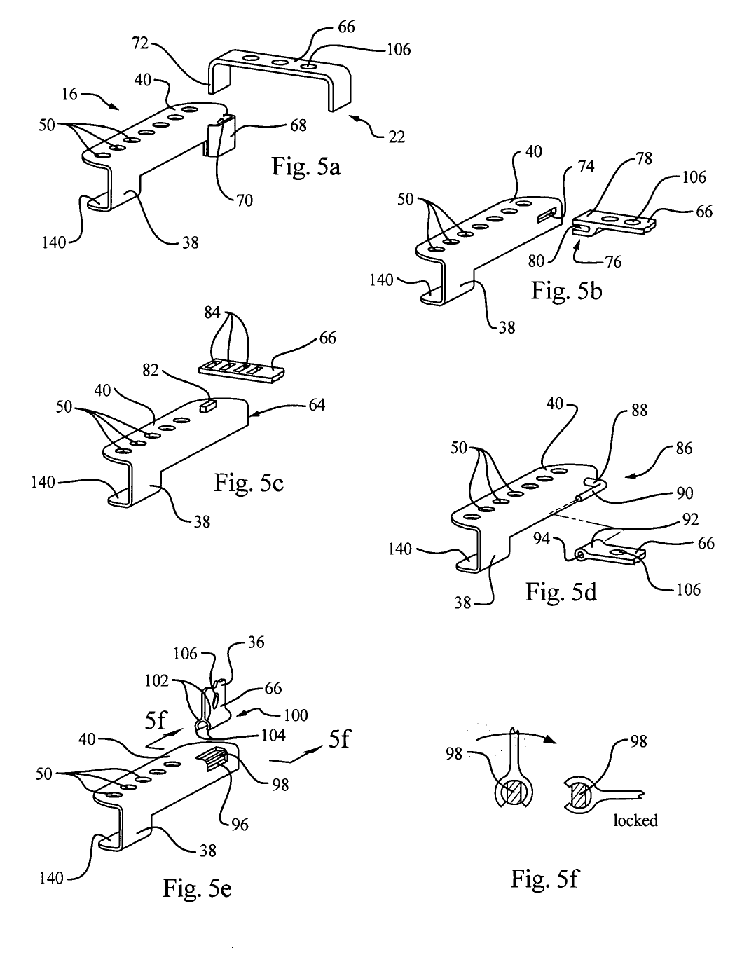

FIGS. 5a through 5f illustrate various embodiments of mechanical-type brace-to-blade releasable attachment means. It should be understood that the embodiments shown in FIGS. 5a through 5f are only shown by way of example and that other types of mechanical brace-to-blade releasable attachment means as well as other types of releasable attachment means such as magnetic and electromagnetic brace-to-blade releasable attachment means may be used without departing from the scope of the present invention.

In the embodiment shown in FIG. 5a, the brace-to-blade releasable attachment means includes a blade tongue 68 extending generally outwardly from the blade first segment 38 substantially adjacent to the blade second segment 40. The blade tongue 68 is provided with a groove 70 formed therein. The spacing brace 22 is provided with a complementary brace tongue 72 extending substantially perpendicularly from the spacing segment 66. The brace tongue 72 is configured and sized for being substantially fittingly and slideably insertable into the groove 70 for releasably attaching the spacing brace 22 to the blade 16.

In the embodiment shown in FIG. 5b, the blade first segment 38 is provided with a coupling aperture 74 formed therein generally adjacent the blade second segment 40. The spacing segment 66 is provided with a complementary brace tongue 76 for engagement with the blade coupling aperture 74. Typically, the brace tongue 76 has a generally forked-shaped configuration defining fork first and second tongues 78, 80. Typically, the fork second tongue 80 is insertable into the blade coupling aperture 74 while the fork first tongue 78 is positionable over at least a portion of the blade second segment 40.

In the embodiment shown in FIG. 5c, the blade second segment 40 is provided with a blade tongue 82 protruding generally outwardly therefrom substantially adjacent one of its longitudinal ends 64. The brace spacing segment 66 is provided with at least one and preferably a set of corresponding spacing segment grooves 84 for substantially fittingly receiving the blade tongue 82. The spacing segment grooves 84 may be strategically positioned along the brace spacing segment 66 for allowing adjustment of the spacing between the blades 16.

In the embodiment shown in FIG. 5d, a generally L-shaped attachment rod 86 extends from the outer surface of the blade first segment 38. The attachment rod 86 defines a rod first or spacing segment 88 extending generally perpendicularly from the blade first segment 38 and a rod second or attachment segment 90 extending in a generally parallel and spaced relationship relative to the blade first segment 38. The brace spacing segment 66 is provided with a brace sleeve 92 formed substantially adjacent at least one of its longitudinal ends. The brace sleeve 92 has a brace sleeve channel 94 extending therethrough for substantially fittingly and slidably receiving the rod second segment 90.

In the embodiment shown in FIG. 5e, the blade 16 has a blade recess 96 formed therein. Typically, the blade recess 96 is formed adjacent the intersection of the blade first and second segments 38, 40. An attachment bar 98 extends across the blade recess 96 in a substantially parallel relationship to the blade longitudinal axis. As illustrated more specifically in FIG. 5f, the attachment bar 98 typically has a generally rectangular cross-sectional configuration. For example, one such generally rectangular cross-section includes two generally opposed flats and two generally opposed concave arc portions. The brace spacing segment 66 is provided with an attachment mouth 100 extending from at least one of its longitudinal ends. The attachment mouth 100 is provided with a pair of attachment jaws 102. The attachment jaws 102 typically have a generally arcuate cross-sectional configuration defining attachment spacing 104 therebetween.

As shown more specifically in FIG. 5f, the attachment jaws 102 and the attachment bar 98 are typically configured and sized so as to allow the attachment bar 98 to slide through the attachment spacing 104 when the brace spacing segment 66 is in a generally perpendicular relationship relative to the blade second segment 40 and in a generally parallel relationship relative to the blade first segment 38. The attachment jaws 102 and the attachment rod 98 are also configured and sized so as to prevent the attachment rod 98 from being withdrawn or retracted from the attachment mouth 100 when the brace spacing segment 66 is in a generally parallel relationship relative to the blade second segment 40 and in a generally perpendicular relationship relative to the blade first segment 38.

As shown in FIGS. 1 through 5f, the brace spacing segment 66 is typically provided with at least one and preferably a set of bracket attachment apertures 106 extending therethrough. As will be hereinafter disclosed in greater details, both the blade and brace attachment apertures 50, 106 may be used for releasably securing a membrane or skin patch 108 thereto. It should be understood that although the blade and brace aperture 50, 106 are shown as having a generally disc-shaped configuration, the blade and brace attachment apertures 50, 106 could have other configurations without departing from the scope of the present invention. Also, as shown in FIG. 5c, the brace attachment apertures 106 may be replaced by apertures 84 adapted to collaborate in both the attachment of the brace spacing segment 66 to a corresponding blade 16 and in the attachment of a membrane 108 to the spacing brace 22.

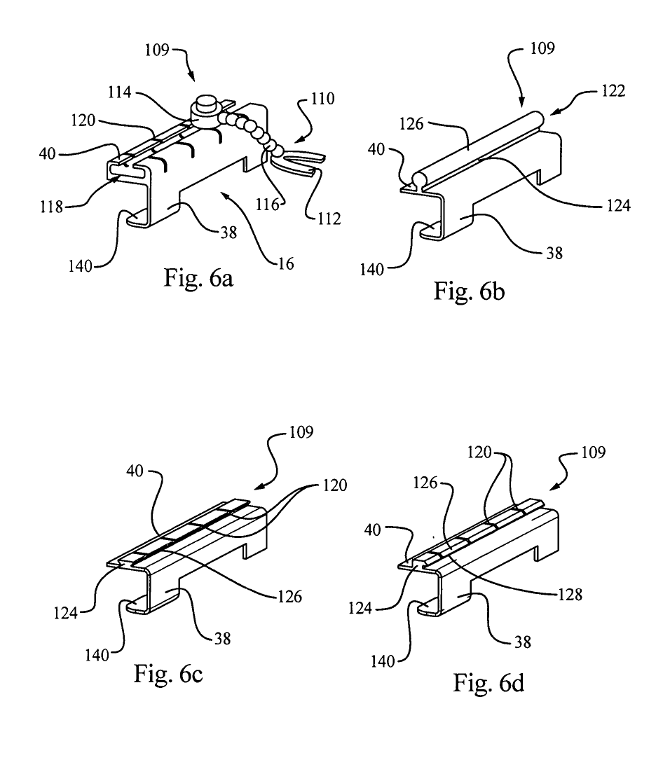

Referring now more specifically to FIGS. 6a through 6d, there are shown portions of blades 16 in accordance with alternative embodiments of the invention wherein the blades 16 are provided with mounting means 109 for allowing mounting thereto of various types of surgical implements. FIG. 6a illustrates a situation wherein the mounting means 109 is used for mounting a heart stabilizer 110 including a generally U-shaped implement abutting plate 112 for abuttingly contacting a section of the target heart. The illustrated heart stabilizer also includes an implement mounting component 114 including a mounting component prong (not shown) extending therefrom for attachment to the mounting means 109 and an implement spacing arm 116 for mechanically coupling the implement abutment plate 112 to the implement mounting component 114 with a predetermined spaced relationship therebetween.

Although FIG. 6a illustrates a situation wherein the surgical implement is used for stabilizing a portion of a beating heart, it should be understood that the mounting means 109 could be used for mounting any suitable type of surgical implement without departing from the scope of the present invention.

In the embodiment shown in FIG. 6a, the mounting means 109 includes a mounting groove, slot, rail or channel 118 extending substantially longitudinally along the blade second segment 40. The mounting channel 118 is configured and sized for substantially fittingly and slidably receiving the mounting prong (not shown) part of the implement mounting component 114 so as to allow slidable movement thereof along the mounting groove 118. In FIG. 6a, the mounting groove 118 is shown as having a generally inverted T-shaped cross-sectional configuration. The T-shaped cross-sectional configuration forms mounting pads which can be gripped by a number of appropriately constructed mounts.

The blade 16 shown in FIG. 6a is also provided with organizing means adapted to be used for organizing or capturing various sutures in the course of a particular surgery. The organizing means typically includes organizing slots or stays 120 formed in the blade second surface 40 and extending substantially perpendicularly relative to the mounting slot 118. In some surgeries such as during a typical coronary artery bypass graft procedure, certain sutures such as pericardial sutures used to position the heart are placed near the beginning of the procedure. In some other surgeries, such as during a typical valve surgical procedure, valve securing sutures used to secure a replacement valve or annuloplasty ring, are placed near the middle or end of the procedure. Accordingly, the sutures stays 120 are typically positioned in a manner that does not interfere with subsequent procedures and instruments. Preferably, the suture stays 120 are positioned such that the placing and manipulating of the sutures or the various instruments and instrument mounts employed during the surgery can be accomplished without interfering with each other.

In the embodiment shown in FIG. 6b, the mounting means which includes a generally elongated mounting protrusion 122 protruding generally outwardly from the blade second surface 40. The mounting protrusion 122 typically includes a protrusion spacing segment 124 extending from the blade second surface 40 and a protrusion retaining segment 126 extending from the protrusion spacing segment 124.

The protrusion retaining segment 126 is typically configured and sized for slidably receiving a generally complementary shaped implement mounting channel (not shown) slidably mounted thereon. In the embodiment shown in FIG. 6b, the protrusion retaining segment 126 has a generally disc-shape cross-sectional configuration. In the embodiment shown in FIG. 6c, the protrusion retaining segment 126 has a generally rectangularly-shaped cross-sectional configuration. In the embodiment shown in FIG. 6d, the protrusion retaining segment 126 has a generally polyhedral configuration defining a retaining lip 128 having a generally triangular cross-sectional configuration.

Typically, both the mounting rail 118 and the mounting protrusion 122 may be generally rectilinear, curved or display a combination of rectilinear and curved segments. In at least one embodiment of the invention (not shown), at least a portion of the mounting rail 118 or mounting protrusion 122 is curved in a manner which substantially follows the profile of the incision opening 14. In a curved configuration, implements extending perpendicular to a generally central axis of the mounting rail 118 or protrusion 122 will naturally point more toward the central area between the blades 16 and, thus, require less positional adjustments or manipulation from their normal, or natural position. It should be understood that both the mounting channel 118 and the mounting protrusions 122 could have various other cross-sectional and/or longitudinal configurations without departing from the scope of the present invention.

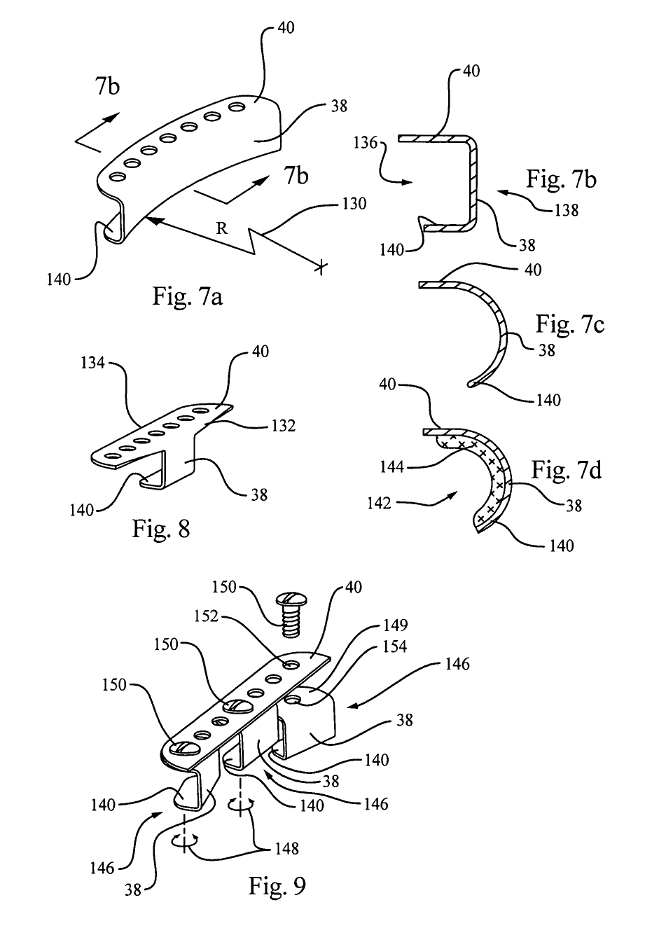

Referring now more specifically to FIGS. 7 through 9, there are shown various possible configurations of the blade 16. In the embodiment shown in FIG. 7a, at least one of the blades 16 has a generally longitudinally curved configuration defined by a blade radius of curvature 130. Alternatively, the blade 16 may have a generally rectilinear or otherwise longitudinally shaped configuration without departing from the scope of the present invention.

Also, in some embodiments such as shown in FIG. 8, an inner longitudinal peripheral edge 132 of the blade second segment 40 may be differently shaped then an outer longitudinal edge 134 of the blade second segment 40. In the embodiment shown in FIG. 8, the inner longitudinal peripheral edge 132 has a generally rectilinear configuration while the outer longitudinal edge 134 has a generally arcuate configuration. It should be understood that both the inner and outer longitudinal edges 132, 134 could have other configurations without departing from the scope of the present invention.

As shown more specifically in FIGS. 7b through 7d, each blade 16 typically has the form of a channel or the like preferably having a generally U-shaped, curved shape or otherwise suitable shape for engaging the incised sternum. Preferably, each blade 16 defines a generally concave interior profile 136 for engaging and holding the sternum and a corresponding convex exterior profile 138. The exterior profile 138 is typically relatively smooth so as to prevent snagging of sutures, interference with other surgical instruments or other types of difficulties.

The blades 16 are typically designed so as to have sufficient strength to withstand the load required to spread the sternum and yet maintain a suitably low profile to facilitate easy insertion into the incision opening 14 and to require as little space within the working incision 14 as possible. When the retractor 10 is used to gain access to the thoracic cavity, a force of substantial magnitude must typically be generated to create the desired incision opening 14. For example, a separating force in the order of 100 pounds may be required to be generated at each blade 16 to achieve the desired separation of a particular sternum. When the blades 16 are made out of a suitable polymeric resin such as a glass filled thermoplastic polyurethane resin or the like, it may be desirable to provide a reinforcing member (not shown) to insure that the blade 16 will not break or otherwise be rendered inoperable as a result of the loads encountered during use.

FIG. 7b illustrates a situation wherein at least one of the blades 16 has a generally “squared” and tilted U-shaped cross-sectional configuration including a generally flat blade first segment 38 and generally flat blade second and third segments 40, 140 extending generally perpendicularly therefrom. In the embodiment shown in FIGS. 7c and 7d, the blade first, second and third segments 38, 40, 140 all have a generally rounded contour integrally with each other so as to define a generally arcuate C-shaped cross-sectional configuration.

As shown more specifically in FIG. 7d, at least one of the blades 16 may be further provided with cushioning means 142 for cushioning the contact between the tissue edges 12 and the blade 16. The cushioning means 142 may be used to reduce trauma to the tissue peripheral edges 12, increase the frictional force or traction between the tissue edges 12 and corresponding blades 16 or both.

The cushioning means may take any suitable form, and thereby adapt to distribute more evenly the separating force over the sternal edges which may be irregularly shaped. FIG. 7d illustrates a situation wherein the cushioning means includes a thin pad or layer 144 of atraumatic and/or friction or non-slip material fixed by way of an adhesive or other suitable fastening technique to the interior surface of at least part of the interior profile 136. The atraumatic or friction enhancing material may be any suitable material including a suitable polymeric or elastomeric resin. Alternatively, the cushioning means 142 may be integrally fabricated into the blades 16. For example, when the blades 16 are injection-moulded components, traction features such as indentations, ribs, raised bumps or the like may be moulded as integral features of the interior profile 136.

FIG. 7a illustrates a situation wherein the blade first and third section segments 38, 140 extends substantially along the full length of the blade second segment 40. Alternatively, as shown in FIG. 8, the blade first and/or third segments 38, 140 may extend only partially along the length of the blade second segment 40.

In yet another embodiment of the invention shown in FIG. 9., the blade first and third segments 38, 140 are provided by blade portions 146 mounted along the length of the blade second segment 40. In the embodiment shown in FIG. 9, the blade second segment takes the form of a generally flat and elongated plate while blade portions 146 have a generally U-shaped configuration defining a blade portion attachment segment 146 for attachment to the blade second segment 40.

Optionally, the blade portions 146 may be pivotally mounted relative to the plate second segment 40 so as to allow pivotal movement therebetween such as indicated schematically by arrows 148. In the embodiment shown in FIG. 9, the blade portions 146 are releasably and pivotally coupled to the plate of the blade second segment 40 using conventional fastening means such as bolts, rivets or screws 150 extending through mounting apertures 152, 154 formed respectively in the blade second segment 40 and in the blade portion attachment segment 146.

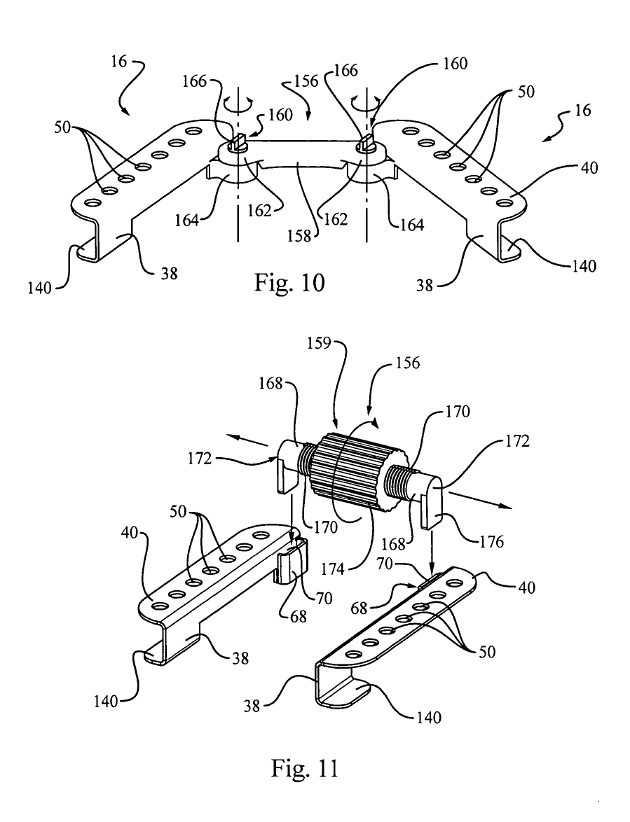

Referring now more specifically to FIGS. 10 through 12b, there are shown alternative embodiments of the invention wherein the driving and bracing means 18, 20 are combined into a driving-bracing means 156 for both moving the blades 16 relative to each other and bracing the blades 16 in a retracted configuration while providing improved visual and physical access to the incision opening 14 when in the braced configuration.

In the embodiment shown in FIG. 10, the driving-bracing means 156 includes a actuating-bracing arm 158 having a pivotal connecting means 160 adjacent at least one and preferably both of the driving-bracing arms longitudinal ends. Each pivotal connecting means 160 may take any suitable form. In the embodiment shown in FIG. 10, the pivotal connecting means 160 includes a ratchet-type mechanism having first and second ratchet members 162, 164 respectively extending from the driving-bracing arm 158 and the corresponding blade 16. The first and second bracket members 162, 164 are provided with cooperating ratchet teeth in operational engagement for providing selective ratchet action in one direction only. A knob type component 166 may be used for allowing an intended user to choose the rotational orientation of the ratchet locking action.

The embodiment shown in FIG. 10 changes the relative position between the blades 16 through a pivotal motion of at least one of the blades 16 relative to a common actuating-bracing arm 158. Hence, the spacing between the blades 16 is changed by modifying the angular relationship between the blades 16. FIGS. 11 and 12 illustrate embodiments of the invention wherein the length of the actuating-bracing arm 159 is changed in order to change the position of the blades 16 relative to one another.

The actuating-bracing arm 159 of the embodiment shown in FIG. 11 includes a pair of arm segments 168 each defining a proximal threaded end 170 and a distal connectable end 172. A threaded sleeve 174 is threadably coupled to the proximal threaded end 170 of at least one and preferably both arm segments 168 so as to vary the effective length of the actuating-bracing arm 159.

The distal end 172 of each arm segment 168 defines an attachment tongue 176 for releasable insertion into an attachment groove such as groove 70 formed in the blade tongue 68. Optionally, a plurality of arm segments 168 threadably linked by threaded sleeves 174 may be used in a serial manner to increase the range of motion of the blades 16 relative to one another. Optionally, a pair of generally opposed driving-bracing arms 159 may be operably connected to each of blades 16, preferably at terminal ends thereof, said attachment tongue 176 and groove 70 being adequately configured to permit selective spacing apart of one or both terminal ends of blades 16.

FIGS. 12a and 12b illustrate an embodiment of the invention wherein the actuating-bracing arm 156 includes at least a pair of plate or arm-segments 175 hinged together by a plate hinge 178. Each plate segment 175 is also hingedly coupled to a corresponding blade 16 using a suitable arm-to-blade hinge connection 180.

A generally L-shaped rod such as rod 86 including a rod spacing segment 88 and a rod-mounting segment 90 may extend from the blade first segment 38 to act as a hinge pin for the blade-to-arm hinged connection 180. A tab section 182 extending from one of the arm segments 175 is adapted to act as an abutment-type stopping means for maintaining the arm segments 175 in a generally co-planar relationship relative to each other when the actuating-bracing arm 156 is in its fully extended configuration shown in FIG. 12b, after a force 179 is applied to the arm 156 at a location proximal to tab section 182. In this fully extended configuration, the plate hinge 178 rests generally below the plane containing hinged connections 180, thereby maintaining the spread apart relationship between blades 16.

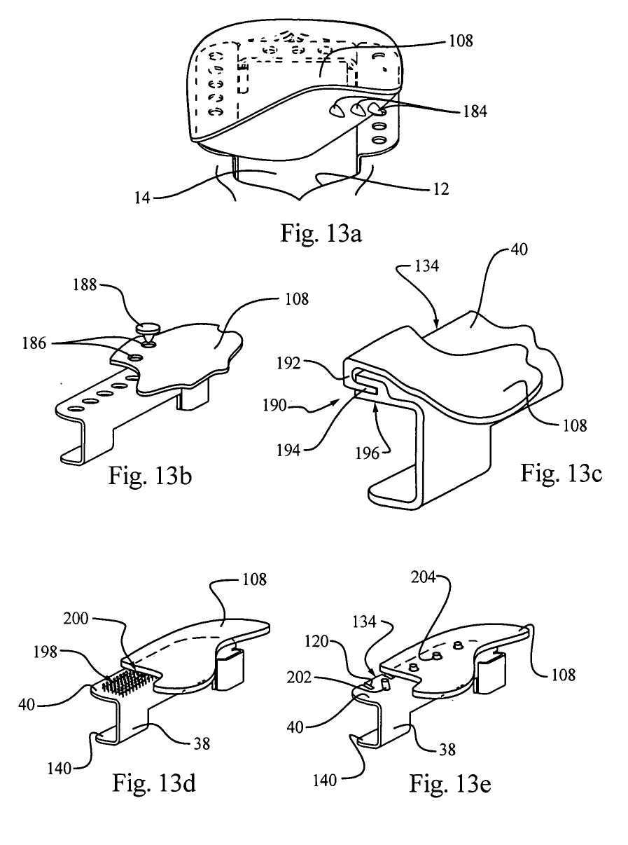

Referring now more specifically to FIGS. 13a through 13e, there is shown yet still another feature of the present invention. The retractor 10 may be provided with a covering component 108 releasably or permanently attachable thereto for at least temporarily extending at least partially across the incision opening 14 so as to at least partially protectively cover the latter.

The retractor 10 may be provided with a membrane-to-blade and/or membrane-to-bracing member attachment means for releasably or permanently attaching the membrane 108 to either one or both of the blades 16 and/or to the bracing component 22.

In situations wherein the blades and/or the bracing components 16, 22 are provided respectively with blade and/or bracing component attachment apertures 50, 106, the membrane 18 may be provided with membrane anchoring protrusions 184 extending from at least one of its surfaces for substantially fittable insertion into corresponding attachment apertures 50, 106.

Alternatively as illustrated in FIG. 13b, the membrane 108 may be provided with membrane attachment apertures 186 extending therethrough substantially adjacent the membrane peripheral edge. The membrane attachment apertures 186 are typically configured and sized so as to be positionable substantially in register with the blade and/or bracing component attachment apertures 50, 106 so as to allow the substantially fitting insertion of attachment component 188 thereinto.

In yet another alternative embodiment of the invention shown in FIG. 13c, the peripheral edge of the membrane 108 is provided with a generally L-shaped attachment flange 190 depending therefrom. The attachment flange 190 typically includes a flange spacing segment 192 extending generally perpendicularly from the membrane 108 and a flange anchoring segment 194 extending inwardly and generally perpendicularly from the flange spacing segment 192 in a substantially spaced and parallel relationship relative to the membrane 108.

The blade second segment 40 is typically provided a forked flange 196 extending from its outer peripheral edge 134. The forked flange 196 is configured and sized for receiving the membrane flange anchoring segment 194.

FIG. 13d illustrates yet another alternative embodiment of the invention wherein the outer surface of the blade's second segment 40 and the peripheral edge of the inner surface of the membrane 108 are provided with cooperative strips 198, 200 of miniature hook and loop fibre such as commercially sold under the trade mark “VELCRO”. Alternatively, cooperative strips 198, 200 may be replaced with adhesive coating surfaces, tape surfaces, or other like surfaces that allow attachment and detachment of membrane 108 to blades 16 or to bracing members 22.

Referring now more specifically to FIG. 13e, there is shown yet another alternative embodiment of the invention wherein the membrane attachment means includes anchoring pins or pegs 202 extending outwardly from the outer surface of the blade second segment 40. The membrane 108 is provided with corresponding pin receiving apertures 204 extending therethrough substantially adjacent its peripheral edge for substantially fittingly receiving the anchoring pins 202. Typically, the anchoring pins 202 are angled relative to the blade second surface 40 so as to extend generally towards the blade outer peripheral edge 134 in a direction leading away from the surface of the blade second segment 40. Alternatively, suture slots or stays 120 which serve during the surgery to retain or organize surgical sutures, may also serve as membrane anchoring slots used to attach said membrane 108. The peripheral edge of membrane 108 may be fittably inserted into said slots 120, preferably with the aid of a surgical suture which urges membrane into fitting engagement therewithin.

The surgical retractor, or component part thereof, defined in the embodiments above may be advantageously manufactured from a surgical grade lightweight material, such as titanium. Such a surgical retractor is especially well-suited for pediatric surgery when it is desirable to have a low weight retractor resting on the chest cavity of an infant or newborn.

We claim:

1. A surgical retractor for retracting a pair of substantially opposed tissue edges defining a surgical opening extending substantially in a surgical opening plane, said retractor comprising:
a pair of tissue engaging members for contacting said tissue edges;
actuating means releasably attachable to said blades for allowing selective adjustment of the spacing between said tissue engaging members; said tissue engaging members being positionable by said actuating means in a retracted configuration wherein they substantially retract said tissue edges away from each other;
bracing means attachable to said blades for maintaining said blades in said retracted configuration when said actuating means is separated from said tissue engaging members.

2. A surgical retractor as recited in claim 1 wherein said bracing means is releasably attachable to said blades.

3. A surgical retractor as recited in claim 1 wherein said actuating means protrudes from said surgical opening by an actuating means protruding distance when operatively coupled to said blades and said bracing means protrudes from said surgical opening by a bracing means protruding distance when operatively coupled to said blades; said actuating means protruding distance being greater then said bracing means protruding distance.
